(19) United States
(12) Patent Application Publication
(10) Pub. No.: US 2024/0321437 A1
(43) Pub. Date: Sep. 26, 2024

(54) MANAGEMENT METHOD AND MANAGEMENT SYSTEM

(71) Applicant: ISUZU MOTORS LIMITED, Yokohama-shi (JP)

(72) Inventor: Mayumi IWAO, Fujisawa-shi (JP)

(21) Appl. No.: 18/603,517

(22) Filed: Mar. 13, 2024

(30) Foreign Application Priority Data
Mar. 20, 2023 (JP) 2023-044139

(51) Int. Cl. G16H 40/20; A61B 5/18

(52) U.S. Cl. CPC G16H 40/20; A61B 5/18

(57) ABSTRACT

A management system of the present disclosure includes: an operation information acquisition section that acquires information on an operation time from a start of operation to an end of the operation of a driver, a nap determination section that performs determination of a quality of a nap of the driver based on heart-rate frequency distribution during sleep outside the operation time of the driver, heart-rate frequency distribution during awakening of the driver, and a heart rate during the nap of the driver within the operation time; and a presentation section that presents a result of the determination.

### CROSS REFERENCE TO RELATED APPLICATIONS

This application is entitled and claims the benefit of Japanese Patent Application No. 2023-044139, filed on Mar. 20, 2023, the disclosure of which including the specification, drawings and abstract is incorporated herein by reference in its entirety.

### TECHNICAL FIELD

The present disclosure relates to a management method and a management system.

### BACKGROUND ART

Transportation companies owning a large number of vehicles such as trucks are required to manage their drivers to operate the vehicles safely.

An operation management method used by a transportation company or the like includes management of a health condition a driver during work (during driving) by attaching a detachable biological sensor to the driver to obtain biological information such as a heart rate and a body temperature of the driver (e.g., see Japanese Patent Application Laid-Open No. 2002-74599). Such a system makes it possible to increase safety by warning and/or encouraging rest when, for example, the driver feels drowsy during operation.

### CITATION LIST

### Patent Literature

### PTL

Japanese Patent Application Laid-Open No. 2002-74599

### SUMMARY OF INVENTION

### Technical Problem

Incidentally, in the conventional operation management, a driver who drives long distance is given a time to take a nap (temporary sleep). Taking a nap is very important in driving long distance safely because the nap can prevent drowsiness from occurring during driving.

The nap is useful not only as compensation for lack of sleep while asleep, but also as a recovery from a poor physical condition, for example. By way of example, taking a nap is useful when a person is not so drowsy, but feels heavy and/or fatigue in eyes.

However, unlike an ordinary sleep at home, a nap is often taken at irregular times and environments, so that a driver may not sleep well during a nap time and may not have sufficient rest. In a case where the driver fails to sleep well during the nap time, the driver will become drowsy in driving and the operational safety will be thus reduced.

The present disclosure has been made in view of the above point, and provides a management method and a management system each capable of performing management with more improved safety by performing management including a quality of a nap.

### Solution to Problem

An aspect of a management method of the present disclosure includes: acquiring information on an operation time from a start of operation to an end of the operation of a driver; determining a quality of a nap of the driver based on heart-rate frequency distribution during sleep outside the operation time of the driver, heart-rate frequency distribution during awakening of the driver, and a heart rate during the nap of the driver within the operation time; and presenting a result of the determining.

An aspect of a management system of the present disclosure includes: an operation information acquisition section that acquires information on an operation time from a start of operation to an end of the operation of a driver; a nap determination section that performs determination of a quality of a nap of the driver based on heart-rate frequency distribution during sleep outside the operation time of the driver, heart-rate frequency distribution during awakening of the driver, and a heart rate during the nap of the driver within the operation time; and a determination-result presentation section that presents a result of the determination.

### Advantageous Effects of Invention

According to the present disclosure, it is possible to perform management with more improved safety by performing management including a quality of a nap.

### BRIEF DESCRIPTION OF DRAWINGS

FIG. 1 illustrates an overall configuration of an operation management system according to an embodiment;

FIG. 2 is a block diagram illustrating a primary configuration of the operation management system according to the embodiment;

FIG. 3A is a graph indicating frequency distribution of heart rates of a driver during daytime activity (during awakening), during sleep, and during a nap;

FIG. 3B is a graph indicating frequency distribution of heart rates of another driver during daytime activity (during awakening), during sleep, and during a nap;

FIG. 3C is a graph indicating frequency distribution of heart rates of still another driver during daytime activity (during awakening), during sleep, and during a nap;

FIG. 3D is a graph indicating frequency distribution of heart rates of still another driver during daytime activity (during awakening), during sleep, and during a nap;

FIG. 3E is a graph indicating frequency distribution of heart rates of still another driver during daytime activity (during awakening), during sleep, and during a nap;

FIG. 3F is a graph indicating frequency distribution of heart rates of yet another driver during daytime activity (during awakening), during sleep, and during a nap;

FIG. 4A is a diagram provided for describing determination performed by a nap determination section; and

FIG. 4B is another diagram provided for describing determination performed by the nap determination section.

### DESCRIPTION OF EMBODIMENTS

Hereinafter, an embodiment of the present disclosure will be described with reference to the drawings.

### <1>Overall Configuration of Operation Management System

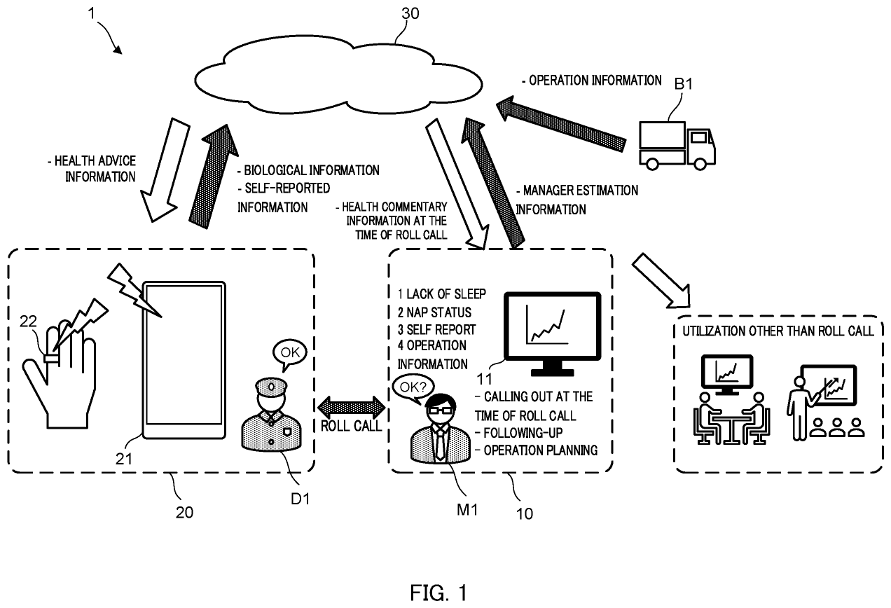

FIG. 1 illustrates an overall configuration of an operation management system in the embodiment.

Operation management system 1 of the present embodiment includes manager side system 10, driver side system 20, and cloud 30.

Manager side system 10 includes terminal 11. Terminal 11 is installed in an office of an operation management department of a transportation company. Terminal 11 is a so-called personal computer and executes an operation required for safe operation management, in response to a manipulation of manager M1.

Terminal 11 displays the following information on driver D1: lack-of-sleep information; nap status information including information on a nap acquisition status and quality of a nap (hereinafter may also be referred to as “nap quality”); self-reported information; operation information; and the like. Manager M1 performs calling out in a roll call and follow-up with driver D1. Manager M1 also makes an operation planning and enters it into terminal 11.

Driver side system 20 includes terminal 21 and biological information sensor 22. In the present embodiment, terminal 21 is a so-called smartphone carried by driver D1, and biological information sensor 22 is a ring-type sensor.

Note that a wristwatch-type sensor may be used as biological information sensor 22, in short, any type sensor may be used as long as biological information of driver D1 can be acquired therefrom including during sleep.

The biological information acquired by biological information sensor 22 is wirelessly transmitted to terminal 21. Biological information sensor 22 acquires information on sleep and information on a nap, as the biological information. Biological information sensor 22 measures heart rates at least during sleep and during a nap.

In the present specification, the term “sleep” means a sleep that a driver takes in sleeping outside an operation time, and the term “nap” means a sleep that the driver takes within the operation time. In general, the “sleep” refers to sleeping on a bed or the like at home on a non-working day, and the “nap” refers to sleeping in a sitting or comfortable posture in a vehicle cabin, a staff room, or the like with the intention of compensating for lack of sleep. Besides, distinguishing whether a sleeping state is “during sleep,” “during a nap,” or “others” can be judged by operation information (whether in working hours and whether in driving).

In terminal 21, an application program for measuring the biological information of driver D1 in cooperation with biological information sensor 22 and an application program for receiving the self-reported information from driver D1 are previously installed. The biological information to be measured by terminal 21 includes, for example, the mean heart rate, the minimum heart rate, the mean respiration rate, a body temperature, and the like, during sleep and during a nap. The biological information of driver D1 accumulates in terminal 21 in time series.

Further, in terminal 21, an application program capable of determining a sleep level of driver D1 based on the biological information of driver D1 may be installed as a health-management application program. Since a method of determining a sleep level based on biological information is known, a detailed description thereof is omitted here. For example, terminal 21 detects falling asleep and waking of driver D1 based on acceleration information from biological information sensor 22. Terminal 21 also determines the sleep level based on the heart rate and the body temperature of driver D1. Terminal 21 or cloud 30 may then calculate a sleep score.

Cloud 30 is configured to be capable of exchanging data wirelessly with manager side system 10 and driver side system 20.

Cloud 30 inputs manager estimation information from manager side system 10. The manager estimation information is information on driver D1 observed by manager M1 at the time of roll call, such as information that driver D1 seems sleepy or unwell.

Cloud 30 also inputs the biological information and the self-reported information from driver side system 20.

Moreover, cloud 30 inputs operation information from vehicle B1 such as a truck owned by the transportation company. The operation information is information acquired by on-board equipment, such as information on an actual time during which vehicle B1 was traveling and information on a travel distance. The operation information may also include, for example, information on drowsiness of a driver based on a line of sight of the driver.

Cloud 30 is configured to include at least a storage apparatus and an information processing apparatus, and generates various types of information by executing a predetermined program using the biological information and the self-reported information input from driver side system 20, the manager estimation information input from manager side system 10, the operation information input from vehicle B1, and the like.

Of the generated various types of information, cloud 30 transmits health advice information to terminal 21 of driver side system 20 and transmits health commentary information at the time of roll call to terminal 11 of manager side system 10. The health advice information and the health commentary information at the time of roll call include information on the sleep and nap of driver D1.

### <2>Primary Configuration of Operation Management System

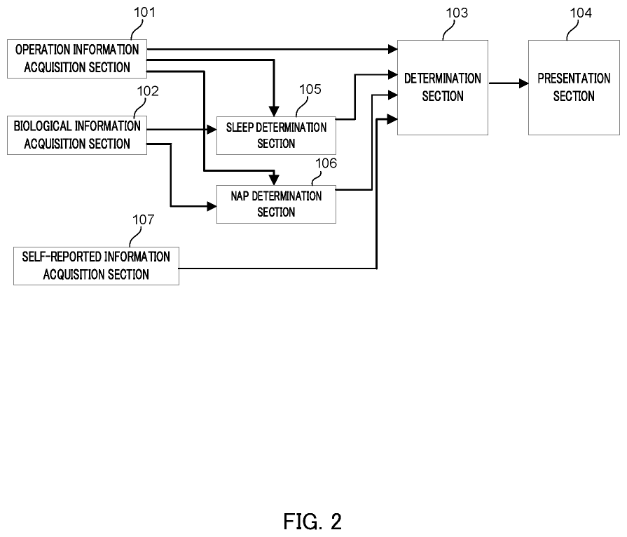

FIG. 2 is a block diagram illustrating a primary configuration of the operation management system according to the present embodiment.

Each element of the functional block illustrated in FIG. 2 is provided on manager side system 10, driver side system 20, or cloud 30 of FIG. 1. For example, operation information acquisition section 101 is provided on manager side system 10, and biological information acquisition section 102 is provided on driver side system 20. Self-reported information acquisition section 107 is provided on driver side system 20. Determination section 103, presentation section 104, sleep determination section 105, and nap determination section 106 are provided on manager side system 10, driver side system 20, or cloud 30.

In practice, manager side system 10, driver side system 20, and cloud 30 are each provided with a Central Processing Unit (CPU), a Read Only Memory (ROM), a Random Access Memory (RAM), and the like. The CPU reads out, from the ROM, a program corresponding to a processing content and loads it to the RAM, thereby realizing the function of each section illustrated in FIG. 2 in cooperation with the loaded program. Incidentally, all or part of the function of each section illustrated in FIG. 2 may be realized by hard-wired circuitry such as an Application Specific Integrated Circuit (ASIC) or a Field-Programmable Gate Array (FPGA). Each functional block of operation management system 1 may be realized by one or more processors, one or more electronic circuitry, or a combination thereof. Operation information acquisition section 101 acquires information on an operation time from a start of operation to an end of the operation of driver D1. The operation information is input by manager M1 via terminal 11 and stored in cloud 30 or terminal 11, for example. Operation information acquisition section 101 acquires the operation information by reading out the stored operation information. Operation information acquisition section 101 acquires the operation information from also communication information (actual record) from vehicle B1.

Biological information acquisition section 102 acquires a heart rate of driver D1 at least during sleep, during a nap, and during awakening. This biological information has been measured by cooperation between terminal 21 and biological information sensor 22, and is stored in terminal 21, cloud 30, or terminal 11.

Sleep determination section 105 determines a quality of sleep (hereinafter may also be referred to as “sleep quality”) of driver D1 based on the heart rates during sleep and during awakening acquired by biological information acquisition section 102. This determination will be described in detail below.

Nap determination section 106 determines a nap quality of driver D1 based on the heart rates during a nap, during sleep, and during awakening acquired by biological information acquisition section 102. This determination will be described in detail below.

Note that, the operation information from operation information acquisition section 101 has been input into sleep determination section 105 and nap determination section 106, and determination section 105 and nap determination section 106 thereby identify, based on the operation information, that a sleep outside an operation time is “sleep” and a sleep in the operation time is a “nap.”

Needless to say, the identification of which sleep is the sleep or the nap is not limited to this example, and various methods are applicable. For example, the sleep and the nap may be identified based on position information or time information. Further, for example, it may be made possible to identify the nap by driver D1 setting terminal 21 to a nap mode. Meanwhile, it can be recognized that driver D1 is awake (in activity) based on the acceleration information of terminal 21, for example.

Determination section 103 inputs determination results of sleep determination section 105 and nap determination section 106. Determination section 103 determines a rest level of driver D1 based on the determination results of both sleep determination section 105 and nap determination section 106 or the determination result of either sleep determination section 105 or nap determination section 106. A determination result made by determination section 103 is output to presentation section 104 and then displayed or presented with a sound by presentation section 104.

Self-reported information acquisition section 107 is an input section that receives a self-report from driver D1, and is embodied by, for example, a touchscreen or a sound input section of terminal 21. Alternatively, self-reported information acquisition section 107 may be embodied by, for example, a touchscreen or a sound input section of terminal 11.

Self-reported information acquisition section 107 acquires, prior to operation, self-reported information on a physical condition and the like from driver D1. The phrase “prior to operation” refers to a predetermined period of time until the start of operation (e.g., within one hour prior to operation) or at the time of roll call. The self-reported information is, for example, a degree of fatigue or physical condition felt by driver D1, or a medicine-taking status.

### <3>Rest Level Determination

Next, a detailed description will be given of determination processing of a rest level according to the present embodiment. In the present embodiment, the determination of the rest level is achieved by sleep determination section 105, nap determination section 106, and determination section 103.

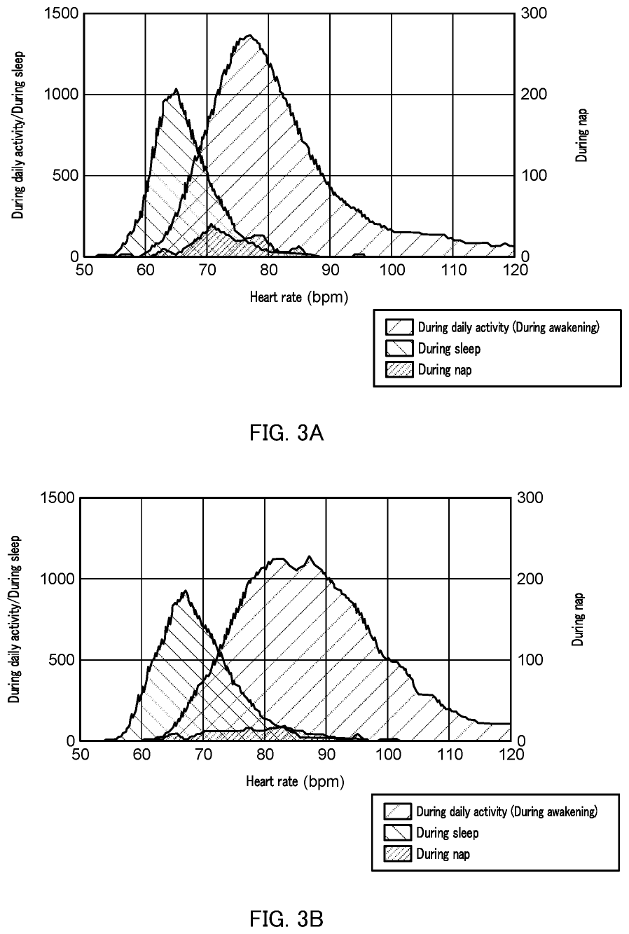

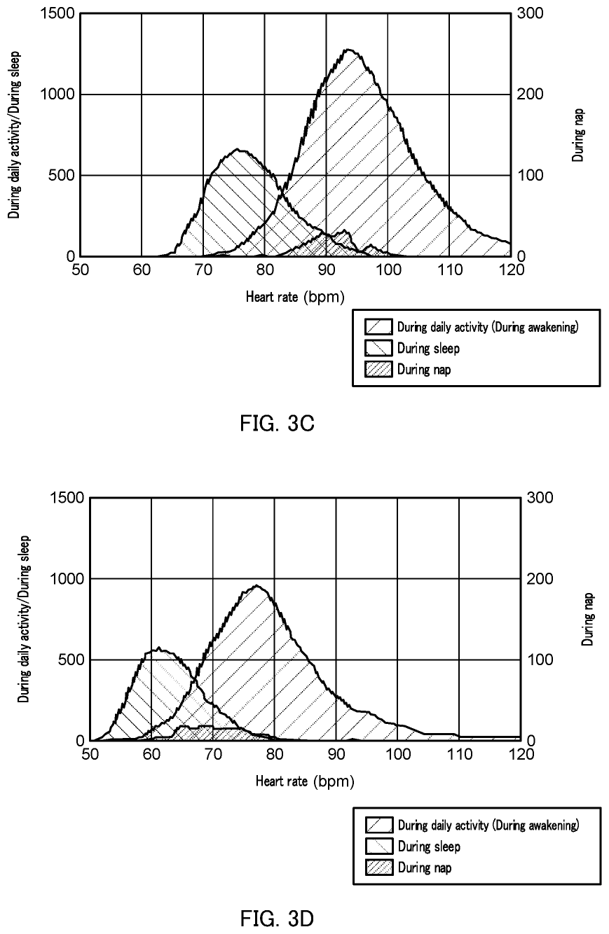

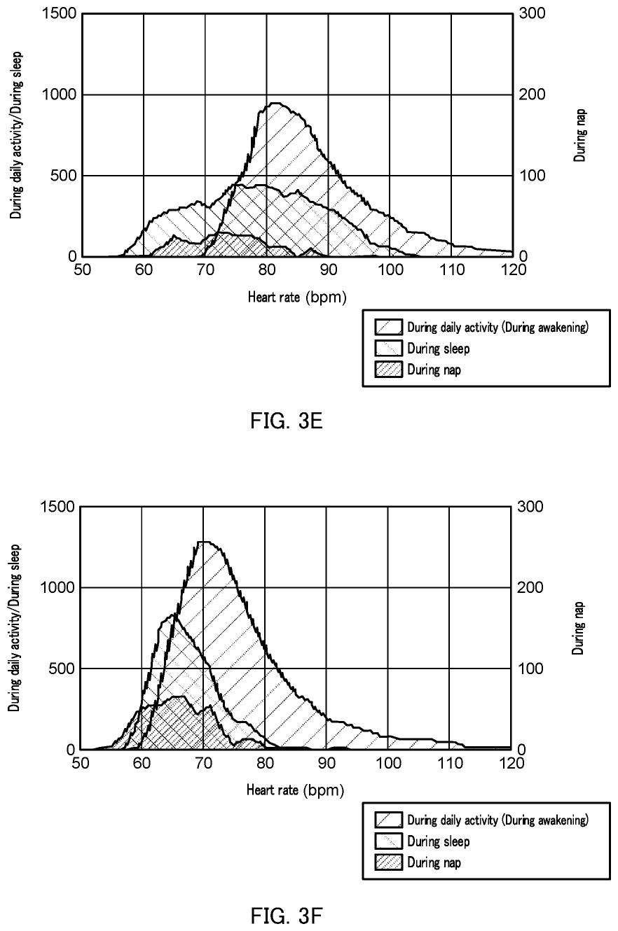

FIGS. 3A to 3F are graphs indicating frequency distribution of heart rates during daytime activity (may be referred to as during awakening), during sleep, and during a nap of six drivers, respectively. In the present embodiment, a rest level of each driver is determined based on this frequency distribution of heart rates during daytime activity (during awakening), during sleep, and during a nap.

The following findings are used in making this determination:
(1) When the heart rates during sleep and during a nap are low, it is likely that good-quality sleep/nap are achieved and a good-quality rest is achieved (rest level is high). By contrast, when the heart rates during sleep and during a nap are high, it is likely that good-quality sleep/nap are not achieved and a good-quality rest is not achieved (rest level is low); and
(2) The frequency distribution during sleep, during a nap, and during awakening is different for each driver.

In the present embodiment, based on the above-mentioned findings (1) and (2), a sleep quality, a nap quality, and a rest level of each driver are determined using the frequency distribution graph for the driver, and thus, a determination result is presented.

The determination of the sleep quality is performed by sleep determination section 105. Sleep determination section 105 determines the sleep quality based on a degree of separation between the frequency distribution graph during sleep and the frequency distribution graph during awakening in a heart rate direction. Specifically, sleep determination section 105 determines that the greater the degree of separation is, the higher the sleep quality is.

The determination of the nap quality is performed by nap determination section 106. Nap determination section 106 compares the frequency distribution graph during a nap with the frequency distribution graph during sleep and the frequency distribution graph during awakening, and then determines the nap quality based on whether the frequency distribution during the nap is similar to the frequency distribution during sleep or the frequency distribution during awakening. Specifically, nap determination section 106 determines that the closer the frequency distribution at the time of nap is to the frequency distribution at the time of sleep (conversely, the farther from frequency distribution at the time of awakening), the higher the nap quality is.

The method of determining a degree of separation by sleep determination section 105 and the method of determining a degree of similarity by nap determination section 106 are not particularly limited, and various methods are applicable.

For example, it may be determined, on the basis of a difference between the heart rate at which the peak of frequency is obtained in the frequency distribution graph during sleep and the heart rate at which the peak of frequency is obtained in the frequency distribution graph during awakening, that the sleep quality is higher as the difference becomes larger.

Additionally, for example, it may be determined, on the basis of a difference between the mean heart rate in the frequency distribution graph during sleep and the mean heart rate in the frequency distribution graph during awakening, that the sleep quality is higher as the difference of the mean heart rates becomes larger.

Further, for example, it may be determined, on the basis of a ratio between an overlapping region and a non-overlapping region in a region surrounded by the frequency distribution graph during sleep and a region surrounded by the frequency distribution graph during awakening, that the sleep quality is higher as the ratio of the non-overlapping region becomes larger.

As for the four drivers indicated in FIG. 3A, FIG. 3B, FIG. 3C, and FIG. 3D, respectively, it can be said that they have the good-quality sleep because the frequency distribution graphs during sleep and the frequency distribution graphs during awakening are separate in the heart rate direction by a predetermined value or more. On the other hand, as for the two drivers indicated in FIG. 3E and FIG. 3F, respectively, it can be said that they do not have the good-quality sleep because the frequency distribution graphs during sleep and the frequency distribution graphs during awakening are not separate in the heart rate direction by a predetermined value or more.

Sleep determination section 105 can select as appropriate which timing to perform the determination. For example, a frequency distribution graph during sleep and a frequency distribution graph during awakening on the day before a roll call day may be used, or, for example, frequency distribution graphs during sleep and frequency distribution graphs during awakening for several days (e.g., one week) before the roll call day may be used.

Alternatively, the determination may be made by comparison of a relationship between a frequency distribution graph during sleep and a frequency distribution graph during awakening immediately before a scheduled operation date of a certain driver relative to a long-term relationship between frequency distribution graphs during sleep and frequency distribution graphs during awakening.

To be more specific, a sleep quality on the day before a roll call day may be determined based on a relationship between heart rate distribution during sleep and heart rate distribution during awakening over a long period of time (one week, one month, three months, one year, or the like) and a relationship between heart rate distribution during sleep and heart rate distribution during awakening on the day before the roll call day.

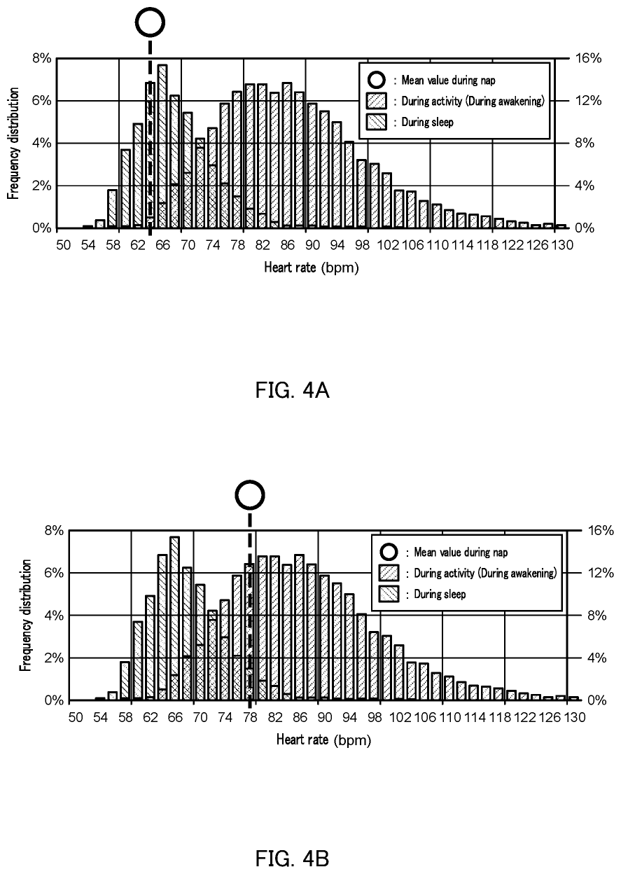

FIG. 4A and FIG. 4B are diagrams provided for describing determination performed by nap determination section 106. Nap determination section 106 compares heart rate distribution during sleep and heart rate distribution during awakening over a long period of time (e.g., one week, one month, three months, one year, or the like) with the mean value of heart rates during this nap. As illustrated in FIG. 4A, when the mean value of heart rates during this nap is close to the heart rate distribution during sleep, nap determination section 106 determines that a high-quality nap had been achieved. On the other hand, as illustrated in FIG. 4B, when the mean value of heart rates during this nap is close to the heart rate distribution during awakening, nap determination section 106 determines that a high-quality nap had not been achieved.

Determination section 103 determines a comprehensive rest level of driver D1 based on the sleep quality obtained by sleep determination section 105 and the nap quality obtained by nap determination section 106. The comprehensive rest level may be determined using a sleep time and a nap time, in addition to the sleep quality and the nap quality. That is, the rest level is high as long as the sleep time and the nap time are sufficiently long even when the sleep quality and the nap quality are low; hence, it is preferable to perform the determination taking this into account.

Further, in the present embodiment, determination section 103 is configured to perform the determination of a rest level using also the self-reported information from self-reported information acquisition section 107. Determination section 103, for example, may determine that the rest level is insufficient upon receiving a self-report such as feeling fatigue from a driver even when a rest level obtained by using the determination results of sleep determination section 105 and nap determination section 106 is high. For example, even when the sleep and nap are sufficient, a rest level may be insufficient due to a large amount of activity during awakening or accumulated fatigue. In such a case, the determination using the self-reported information is effective.

Presentation section 104, for example, presents the following phrases by displaying or sounding: “You may not be well rested with a nap. If you have the opportunity to take a rest next, please lie down and rest as much as possible.”; “The sleep quality was not good. You should take a nap as much as possible.”; or “The rest level is insufficient. You should take a nap.” in operation. Alternatively, a nap may be encouraged by an alarm sound or the like.

### <4>Summary

As described above, according to the present embodiment, operation management system 1 includes: operation information acquisition section 101 that acquires information on an operation time from a start of operation to an end of the operation of driver D1; nap determination section 106 that performs determination a nap quality of driver D1 based on heart-rate frequency distribution during sleep outside the operation time of driver D1, heart-rate frequency distribution during awakening of driver D1, and a heart rate during a nap of driver D1 within the operation time; and a determination-result presentation section (presentation section 104) that presents a result of the determination.

This enables performing operation management including a nap quality, and thus, operation management with more improved safety can be performed.

Operation management system 1 also includes: sleep determination section 105 that determines a sleep quality during sleep of driver D1 based on heart-rate frequency distribution during sleep of driver D1 and heart-rate frequency distribution during awakening of driver D1; and determination section 103 that determines a rest level of driver D1 based on the nap quality and the sleep quality.

This makes it possible to obtain and present a rest level taking into account the nap quality and the sleep quality, and thus, operation management with more improved safety can be performed.

The embodiment described above is merely an example of specific implementation of the present invention, and the technical scope of the present invention should not be restrictively interpreted by this embodiment. That is, the present invention may be implemented in various forms without departing from the spirit thereof or the major features thereof.

### INDUSTRIAL APPLICABILITY

The present disclosure is useful as a technique for safe management performed by a transportation company.

### REFERENCE SIGNS LIST

1 Operation management system
10 Manager side system
11, 21 Terminal
20 Driver side system
22 Biological information sensor
30 Cloud
101 Operation information acquisition section
102 Biological information acquisition section
103 Determination section
104 Presentation section
105 Sleep determination section
106 Nap determination section
107 Self-reported information acquisition section

1. A management method, comprising:
acquiring information on an operation time from a start of operation to an end of the operation of a driver;
determining a quality of a nap of the driver based on heart-rate frequency distribution during sleep outside the operation time of the driver, heart-rate frequency distribution during awakening of the driver, and a heart rate during the nap of the driver within the operation time; and
presenting a result of the determining.

2. The management method according to claim 1, wherein,
in the determining, the quality of the nap is determined based on whether frequency distribution of the heart rate during the nap of the driver within the operation time is similar to the heart-rate frequency distribution during the awakening or the heart-rate frequency distribution during the sleep.

3. The management method according to claim 1, wherein the determining further comprises:
determining a quality of the sleep during the sleep of the driver based on the heart-rate frequency distribution during the sleep of the driver and the heart-rate frequency distribution during the awakening of the driver; and
determining a rest level of the driver based on the quality of the nap and the quality of the sleep.

4. The management method according to claim 3, wherein,
in the determining, it is determined that the quality of the sleep is higher as a difference between a mean heart rate in the heart-rate frequency distribution during the sleep and a mean heart rate in the heart-rate frequency distribution during the awakening becomes larger.

5. The management method according to claim 3,
in the determining, it is determined that the quality of the sleep is higher as a region in which a region surrounded by the heart-rate frequency distribution during the sleep and a region surrounded by the heart-rate frequency distribution during the awakening do not overlap with each other becomes larger.

6. A management method, comprising:
acquiring information on an operation time from a start of operation to an end of the operation of a driver;
determining a quality of sleep during sleep of the driver based on heart-rate frequency distribution during the sleep of the driver and heart-rate frequency distribution during awakening of the driver; and
presenting a result of the determining.

7. A management system, comprising:
an operation information acquisition section that acquires information on an operation time from a start of operation to an end of the operation of a driver;
a nap determination section that performs determination of a quality of a nap of the driver based on heart-rate frequency distribution during sleep outside the operation time of the driver, heart-rate frequency distribution during awakening of the driver, and a heart rate during the nap of the driver within the operation time; and
a determination-result presentation section that presents a result of the determination.

8. A management system, comprising:
an operation information acquisition section that acquires information on an operation time from a start of operation to an end of the operation of a driver,
a sleep determination section that performs determination of a quality of sleep during sleep of the driver based on heart-rate frequency distribution during the sleep of the driver and heart-rate frequency distribution during awakening of the driver; and
a determination-result presentation section that presents a result of the determination.
